(19) United States
(12) Patent Application Publication
(10) Pub. No.: US 2019/0183388 A1
(43) Pub. Date: Jun. 20, 2019

(54) BODY MONITORING AND TRAINING DEVICE

(71) Applicant: Upright Technologies Ltd., Yehud (IL)

(72) Inventors: Oded Cohen, Savyon (IL); Liran Reller, Rehovot (IL); Nir Porat, Jerusalem (IL)

(21) Appl. No.: 16/326,666

(22) PCT Filed: Aug. 6, 2017
(86) PCT No.: PCT/IL2017/050864

§ 371 Date: Feb. 19, 2019

Related U.S. Application Data
(60) Provisional application No. 62/376,467, filed on Aug. 18, 2016.

(51) Int. Cl. A61B 5/11; A61B 5/00

(52) U.S. Cl. CPC A61B 5/1116; A61B 5/4561; A61B 5/486; A63B 23/02; A61B 5/6822; A61B 5/6814; A61B 5/6803

(57) ABSTRACT

A body movement feedback system including at least one position sensor operative to sense in real time at least relative positions of mutually articulated body portions of a user and to provide sensor outputs, a mutually articulated body portion position processor, receiving the sensor outputs of the at least one position sensor and providing processor outputs representing relative movements of the mutually articulated body portions and a real time user feedback generator operative to generate predetermined feedback to the user in response to predetermined selected movements of the mutually articulated body portions.

### REFERENCE TO RELATED APPLICATIONS

Reference is hereby made to U.S. Provisional Patent Application Ser. No. 62/376,467, filed Aug. 18, 2016, entitled: UPPER BACK AND NECK MONITORING AND TRAINING DEVICE, the disclosure of which is hereby incorporated by reference and priority of which is hereby claimed pursuant to 37 CFR 1.78(a).

Reference is also made to PCT Application No. PCT/IB2014/059041, filed Feb. 17, 2014, and entitled POSTURE DETECTION DEVICE, published Aug. 21, 2014 as PCT Published Patent Application WO 2014/125448 A1, the disclosure of which is hereby incorporated by reference.

Reference is also made to PCT Application No. PCT/IL2016/050847, filed Aug. 3, 2016, and entitled BODY MOVEMENT FEEDBACK SYSTEM AND METHOD, the description of which is hereby incorporated by reference and priority of which is hereby claimed pursuant to 37 CFR 1.78(a).

### FIELD OF THE INVENTION

The present invention relates to body motion training systems and methodologies.

### BACKGROUND OF THE INVENTION

There exist various systems and devices for body motion training.

### SUMMARY OF THE INVENTION

The present invention seeks to provide improved systems and devices for body motion training.

There is thus provided in accordance with a preferred embodiment of the present invention a body movement feedback system including at least one position sensor operative to sense at least relative positions of mutually articulated body portions of a user, and particularly the upper back, chest or neck orientation relative to the gravity or relative to the head orientation in real time and to provide sensor outputs, a mutually articulated body portion position processor, receiving the sensor outputs of the at least one position sensor and providing processor outputs representing relative movements of the mutually articulated body portions and a real time user feedback generator operative to generate predetermined feedback to the user in response to predetermined selected movements of the mutually articulated body portions.

In accordance with a preferred embodiment of the present invention the body movement feedback system also includes a user feedback selector adapted to receive feedback selection inputs from a person and operative to assign selectable user feedbacks to selectable movements of the mutually articulated body portions.

Preferably, the at least one position sensor includes at least two body portion orientation sensors attached to the upper back, chest or neck and the head. Alternatively, the at least one position sensor includes at least one body portion orientation sensor attached to the upper back, chest or neck and the head.

Preferably, the predetermined feedback includes at least one of tactile feedback, auditory feedback and visual feedback.

In accordance with a preferred embodiment of the present invention the at least one position sensor includes at least one first sensor mounted onto a user's upper back, chest or neck preferably by a necklace, headset (as LG TONE, SOL REPUBLICK Shadow) or headphone (as LG TONE, SOL REPUBLICK Shadow) that lays on the user's neck at a first location. In an alternative embodiment of the present invention the at least one position sensor includes at least one first sensor mounted onto a user's upper back, chest or neck preferably by a necklace, headset (as LG TONE, SOL REPUBLICK Shadow) or headphone (as LG TONE, SOL REPUBLICK Shadow) that lays on the user's neck at a first location and at least a second sensor mounted to the user's head, preferably by an earphone, earring, audio aid device, hat, hair pin, eye-glasses and hair bow at a second location, the first and second locations being mutually separated.

Preferably, the real time user feedback generator includes a feedback transducer operative to provide a real time notification to a user when at least one predetermined orientation is reached.

Preferably, the body movement feedback system also includes a portable controller communicating wirelessly with the microprocessor.

There is also provided in accordance with another preferred embodiment of the present invention a method for body movement feedback including the steps of sensing, preferably the user's upper back, chest or neck relative to gravity and alternatively the user's upper back, chest or neck and head relative positions of a user in real time and providing sensor outputs, receiving the sensor outputs and providing processor outputs representing relative movements and generating predetermined feedbacks to the user in response to predetermined movements.

In accordance with a preferred embodiment of the present invention the method for body movement feedback also includes receiving feedback selection inputs from a person and assigning selectable user feedbacks to selectable movements of the mutually articulated body portions.

In accordance with a preferred embodiment of the present invention the sensing the orientation of at one body portions includes measuring orientation of the upper back, chest or the neck relative to gravity. Alternatively, in accordance with a preferred embodiment of the present invention the sensing the orientation of the upper back, chest or neck and the head includes measuring mutual orientation. Preferably, the predetermined feedback includes at least one of tactile feedback, auditory feedback and visual feedback.

There is further provided in accordance with yet another preferred embodiment of the present invention a body position training device for measuring a user's posture by measuring the upper back, chest or neck orientation including at least one sensor mounted onto a user's upper back, chest or neck preferably by necklace, headset (as LG TONE, SOL REPUBLICK Shadow), headphone (as LG TONE, SOL REPUBLICK Shadow) that lays on the user's neck, a microprocessor receiving inputs from sensor for calculating an orientation of the users posture and a feedback transducer operative to provide a real time notification to a user when a predetermined orientation of the upper back, chest or neck is reached. Alternatively, in another embodiment of the present invention there is provided a body position training device for measuring a user's posture and the orientation of the upper back, chest or neck relative to the head including at least one first sensor mounted onto a user's upper back, chest or neck preferably by necklace, headset (as LG TONE, SOL REPUBLICK Shadow), headphone (as LG TONE, SOL REPUBLICK Shadow) that lays on the user's neck, at least a second sensor mounted to the user's head preferably by an earphone, earring, audio aid device, hat, eye-glasses and hair bow, a microprocessor receiving inputs from the first and second sensors for calculating an orientation of the user's posture and the orientation of the head relative to the upper back, chest or neck and a feedback transducer operative to provide a real time notification to a user when a predetermined orientation is reached.

In accordance with a preferred embodiment of the present invention the body position training device for measuring a user's posture and/or the orientation of the head relative to the upper back, chest or neck orientation also includes a portable controller communicating wirelessly with the microprocessor.

### BRIEF DESCRIPTION OF THE DRAWINGS

The present invention will be understood and appreciated more fully from the following detailed description, taken in conjunction with the drawings in which:

FIG. 1 is a simplified illustration of a body position training system including a body-mounted body position training device and a portable control device in accordance with a preferred embodiment of the present invention;

FIG. 2 is a simplified perspective illustration of the body-mounted body position training device of FIG. 1;

FIG. 3 is a simplified perspective illustration of a body position training device constructed and operative in accordance with another preferred embodiment of the present invention;

FIGS. 4A, 4B and 4C are simplified pictorial illustrations of two phases in the operation of the body position training system for training;

FIGS. 5A, 5B and 5C are simplified pictorial illustrations of two phases in the operation of the body position training system for training;

FIGS. 6A, 6B and 6C are simplified pictorial illustrations of two phases in the operation of the body position training system for training;

FIGS. 7A and 7B are simplified illustrations of an alternative embodiment of the second sensor mounted on a hearing aid device;

FIGS. 8A and 8B are simplified illustrations of an alternative embodiment of the second sensor mounted on glasses;

FIGS. 9A and 9B are simplified illustrations of an alternative embodiment of the second sensor mounted on an earring;

FIGS. 10A and 10B are simplified illustrations of an alternative embodiment of the second sensor mounted on a hair bow; and

FIGS. 11A and 11B are simplified illustrations of an alternative embodiment of the second sensor mounted on a hat.

### DETAILED DESCRIPTION OF PREFERRED EMBODIMENTS

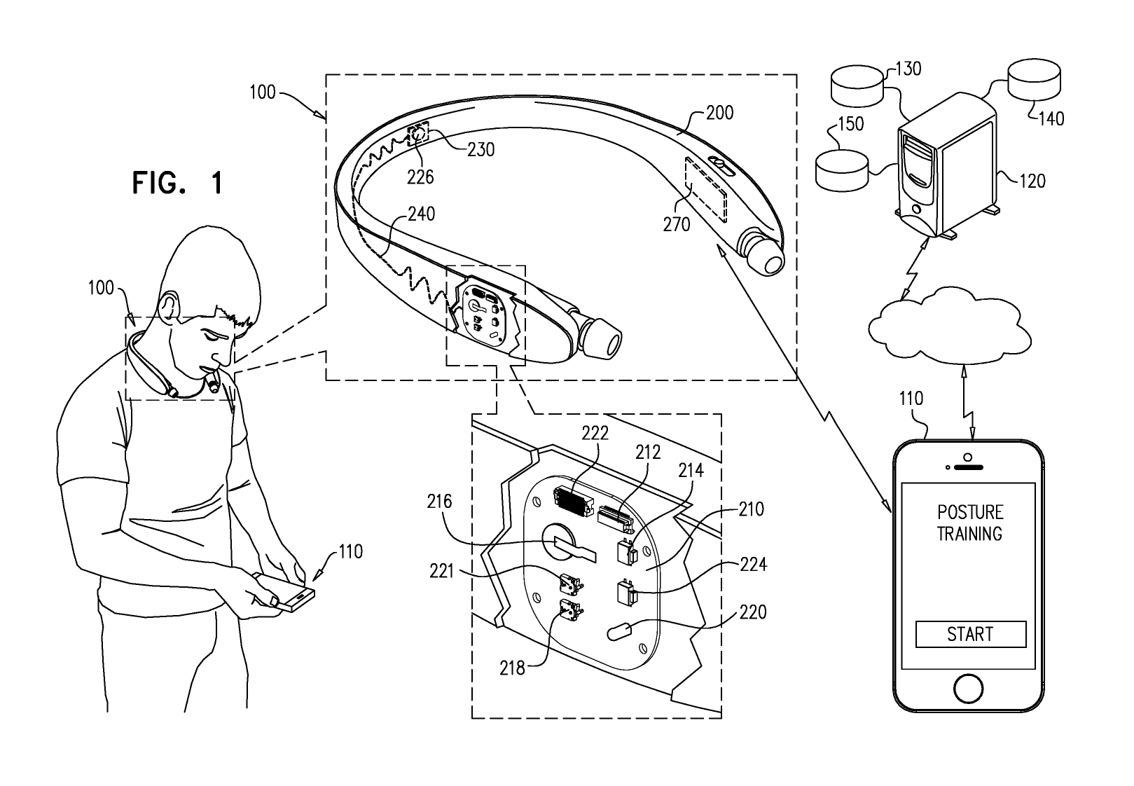

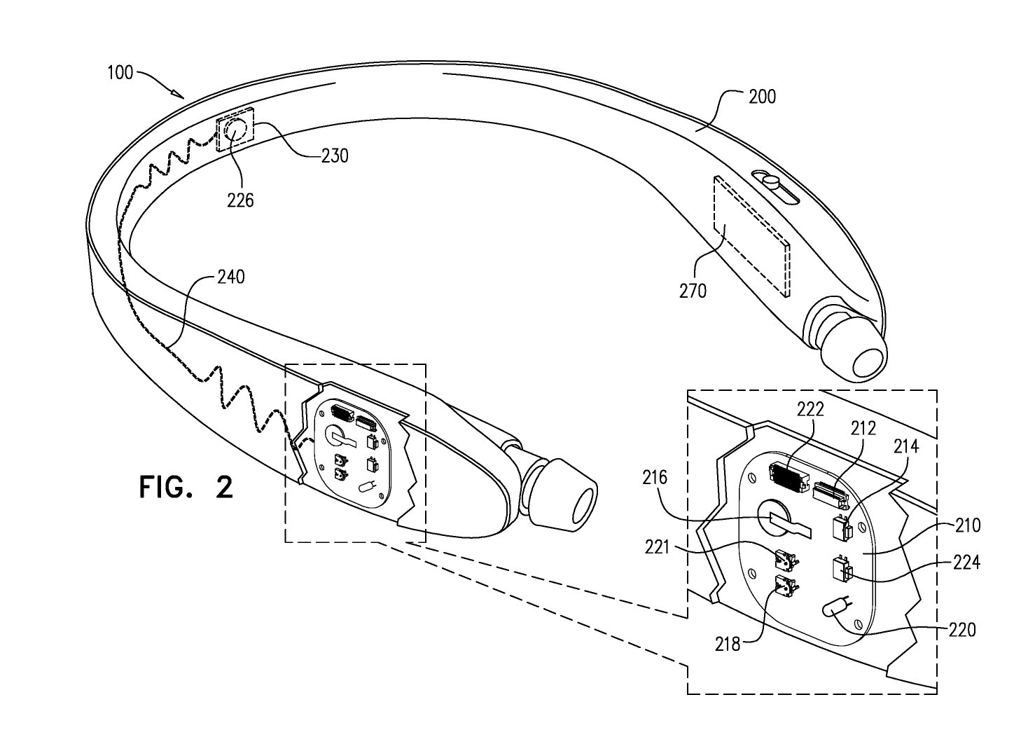

Reference is now made to FIG. 1, which is a simplified illustration of a body position training system including a body-mounted body position training device 100 that is preferably worn by a user and a portable control device 110 that is preferably controlled by the user, and to FIG. 2, which illustrates the body position training device 100 seen in FIG. 1.

The body-mounted body position training device 100 is preferably controlled by portable control device 110, which may be embodied in a generic smartphone, a smartwatch or any other suitable mobile communication device, which may or may not be specifically designed for use in the system.

The portable control device 110 preferably communicates wirelessly with the body-mounted body position training device 100 and wirelessly, typically via the Internet, with a server 120. The user preferably is the only person who can control the body position training device 100.

The server 120 may provide selectable access to one or more of the following: a user database 130, a training program database 140 and an exercise protocol database 150. Training programs from training program database 140 and exercise protocols from exercise protocol data base 150, as well as other data and information, may be sent, via server 120, to portable control device 110, to be carried out by the user.

Programmed commands and conditions related to the assigned training programs and exercise protocols may be sent from the portable control device 110 to the body position training device 100. According to these commands and conditions, the body position training device 100 collects data from the user and administers feedback to the user during monitoring and training. For example, such data and feedback could be related to the user's incorrect head orientation relative to his neck.

Information sent to a user's portable control device 110, such as exercise protocols, training programs and data collected by the body position training device 100, is preferably stored in the user's profile in the user database 130. Furthermore, the aforementioned user profile may preferably be accessed, preferably by the user, via the server 120.

During use of the system, data collected by the body position training device 100 is sent wirelessly by the body position training device 100 to the portable control device 110. From the portable control device 110, information and data preferably is exported to the server 120.

Preferably, the body position training device 100 provides feedback to the user. The body position training device 100 preferably transmits data acquired during the exercises to the portable control device 110, typically for viewing by the user and also, preferably, transmits the data acquired, via the portable control device 110, to the server 120.

As seen in FIGS. 1-2, the body position training device 100 preferably includes an enclosure 200. Enclosure 200 includes the body position training device components described hereinbelow and preferably also includes headphones (such as LG Tone, SOL REPUBLICK Shadow) or any other suitable electronic device.

Disposed within enclosure 200 is a printed circuit board 210, on which are preferably mounted a microprocessor 212, as well as a tactile feedback generator 214, a battery 216, a wireless transceiver 218, a visual feedback generator 220, a memory 221 and an auditory feedback generator 222, all coupled to microprocessor 212. Optionally, an orientation sensor 224 may also be mounted on printed circuit board 210 and coupled to the microprocessor 212. In one embodiment, an auditory compatible device 270 is incorporated within enclosure 200 of the body position training device 100. Preferably, the audio feedback occurs through the auditory compatible device 270 and optionally, feedback may be provided through auditory feedback generator 222.

Preferably, an orientation sensor 226, preferably mounted on a circuit board 230, is also disposed within enclosure 200. As noted above, an additional orientation sensor 224 is optionally mounted on circuit board 210. Circuit board 230 is preferably not rigidly coupled to printed circuit board 210. Circuit boards 230 and 210 are preferably connected via flexible wire conductors 240. Orientation sensors 224 and 226 are preferably model numbers BHI 160 or BMI 160 manufactured by Bosch Sensor Tech from Gerhard-Kindler-StraBe 9 72770 Reutlingen/Kusterdingen, Germany.

Orientation sensors 224 and 226 preferably each include an accelerometer, for measuring the angle relative to gravity, and/or a gyroscope, for measuring the orientation of the body position training device 100.

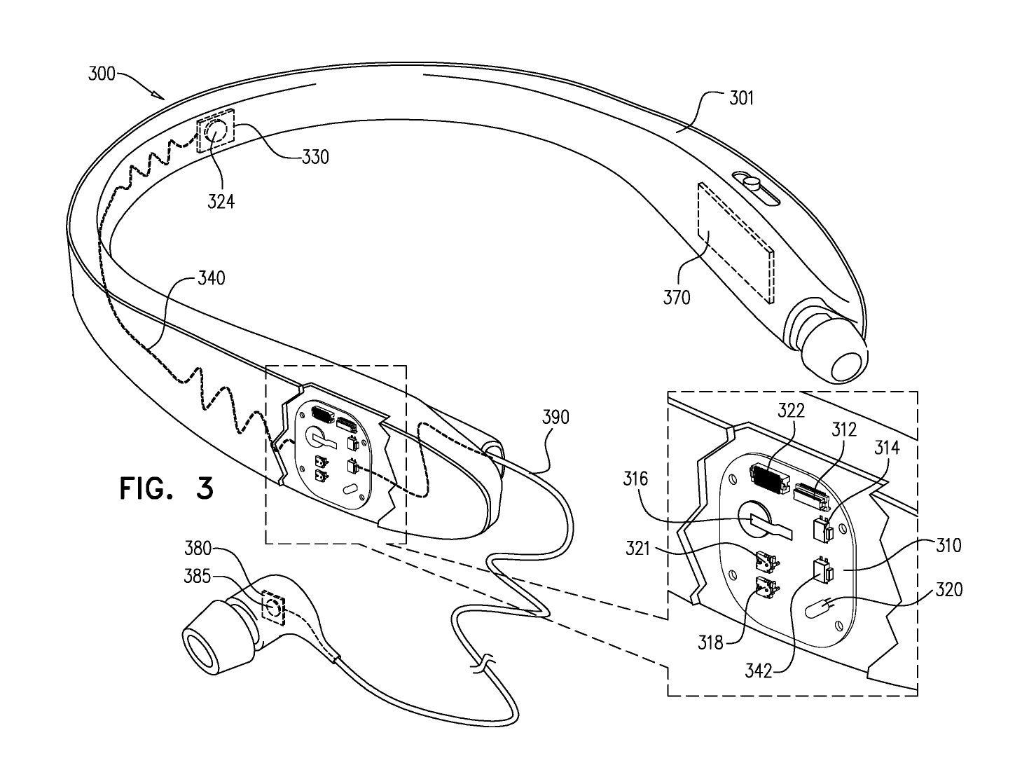

Reference is now made to FIG. 3, which illustrates an alternative embodiment of the body position training device 100 shown in FIG. 1.

As seen in FIG. 3, the body position training device, here designated by reference numeral 300, preferably includes an enclosure 301, which includes body position training device components, as described in detail hereinbelow, and preferably also includes headphone components (such as LG Tone, SOL REPUBLICK Shadow) or any other suitable electronic device components.

Disposed within enclosure 301 is a printed circuit board 310, on which are preferably mounted a microprocessor 312, as well as a tactile feedback generator 314, a battery 316, a wireless transceiver 318, a visual feedback generator 320, a memory 321 and an auditory feedback generator 322, all coupled to microprocessor 312. An orientation sensor 324 is preferably mounted on circuit board 330, which is connected, preferably via conductors 340, to circuit board 310. Optionally, an additional orientation sensor 342 may also be mounted on printed circuit board 310 and coupled to microprocessor 312.

In one embodiment, an auditory compatible device 370 is incorporated within enclosure 301 of the body position training device 300. In this embodiment, the audio feedback preferably occurs through the auditory compatible device 370 and optionally, audio feedback may be provided through auditory feedback generator 322.

Also disposed within enclosure 301 of body position training device 300 is an additional orientation sensor 380, preferably mounted on a circuit board 385, which is not rigidly mounted to circuit board 310 and is located on the head of the user when in use, preferably in an earphone. Circuit board 385 is preferably connected to circuit board 310 via conductors 390. Alternatively, circuit boards 310 and 385 connected via wireless connection. Orientation sensors 324, 342 and 380 are preferably model numbers BHI 160 or BMI 160 manufactured by Bosch Sensor Tech from Gerhard-Kindler-StraBe 9 72770 Reutlingen/Kusterdingen, Germany.

Orientation sensors 324, 342 and 380 preferably each include an accelerometer, for measuring the angle relative to gravity, and/or a gyroscope, for measuring the orientation of the body position training device 100.

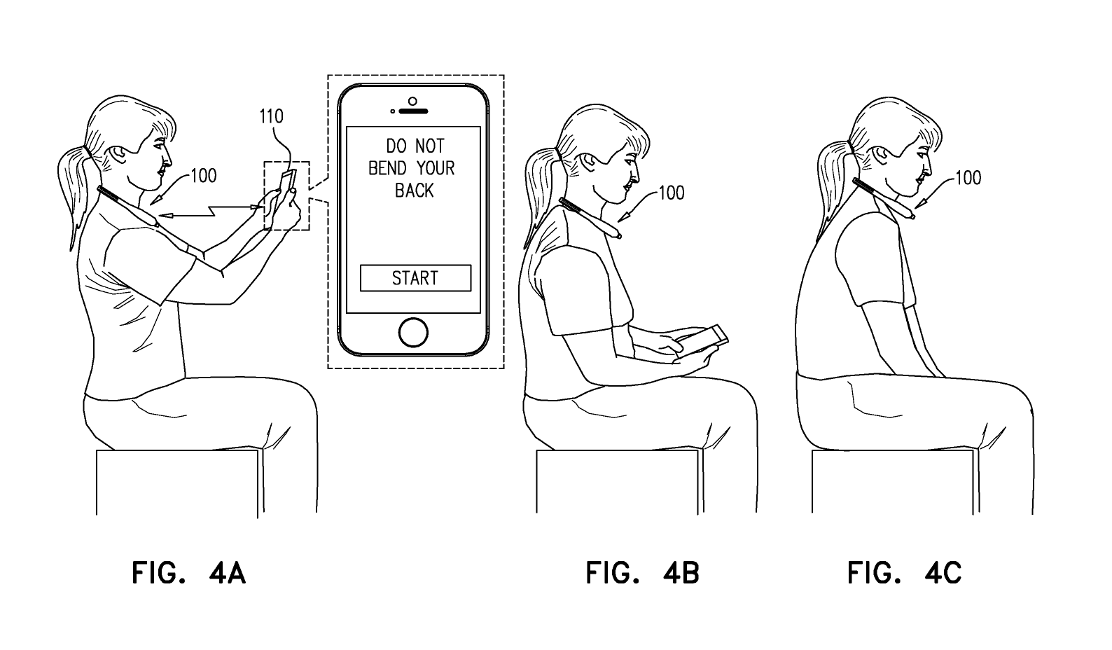

Reference is now made to FIGS. 4A, 4B and 4C, which are simplified pictorial illustrations of the operation of the body position training system for training. For simplicity, the description refers to body position training system of FIGS. 1 and 2 including body-mounted body position training device 100 and portable control device 110.

FIG. 4A illustrates an initial phase in which training instructions are downloaded to portable control device 110.

The training instructions, downloaded to portable control device 110, preferably include instructions to the user, which are intended for display on portable control device 110, as shown, as well as instructions to body-mounted body position training device 100, which are preferably relayed to body-mounted body position training device 100 via the portable control device 110. The instructions to the body position training device preferably include at least one of:
1. Feedback generation instructions based on a predetermined operative orientation of the body-mounted body position training device 100, such as a 30 degree bend of the upper back, as shown in FIG. 4C.
2. Feedback generation instructions based on exceedance of a predetermined operative orientation threshold of the body-mounted body position training device 100, such as a bend of the upper back exceeding 15 degrees, as seen in FIG. 4B.

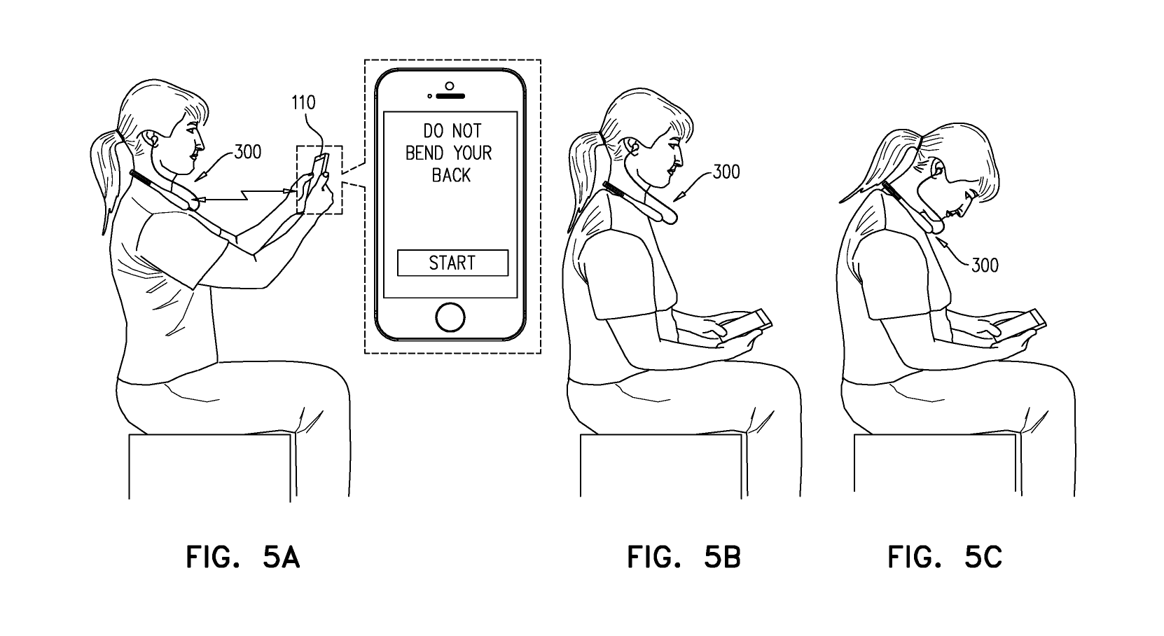

Reference is now made to FIGS. 5A, 5B and 5C, which are simplified pictorial illustrations of the operation of the body position training system for training. For simplicity, the description refers to body position training device 300 shown in FIG. 3 including body-mounted body position training device 300 and portable control device 110.

FIG. 5A illustrates an initial phase in which training instructions are downloaded to the portable control device 110.

The training instructions, downloaded to portable control device 110, preferably include instructions to the user, which are intended for display on portable control device 110, as shown, as well as instructions to body-mounted body position training device 300, which are preferably relayed to body-mounted body position training device 300 via the portable control device 110. The instructions to the body position training device preferably include at least one of:
1. Feedback generation instructions based on a predetermined operative orientation of the body-mounted body position training device 300, such as a 30 degree bend of the upper back, as shown in FIG. 5C.
2. Feedback generation instructions based on exceedance of a predetermined operative orientation threshold of the body-mounted body position training device 300, such as a bend of the upper back exceeding 15 degrees as seen in FIG. 5B.

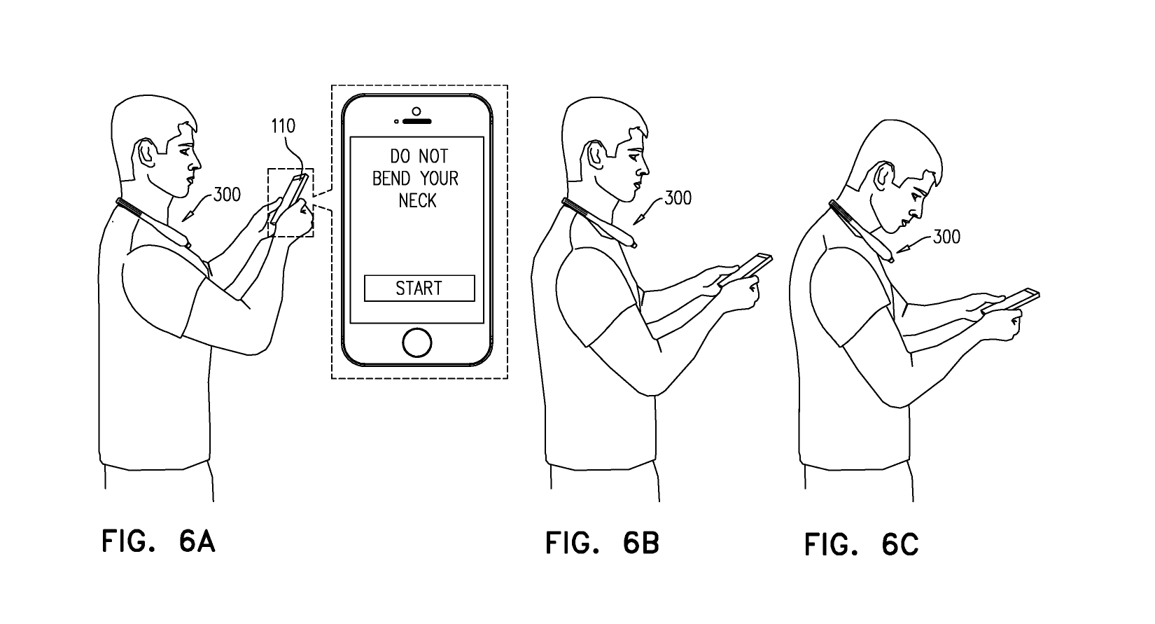

Reference is now made to FIGS. 6A, 6B and 6C, which are simplified pictorial illustrations of the operation of the body position training system for training. For simplicity, the description refers to body position training device 300 shown in FIG. 3 including body-mounted body position training device 300 and portable control device 110.

FIG. 6A illustrates an initial phase in which training instructions are downloaded to the portable control device 110.

The training instructions, downloaded to portable control device 110, preferably include instructions to the user, which are intended for display on portable control device 110, as shown, as well as instructions to the body-mounted body position training device 300, which are preferably relayed to body-mounted body position training device 300 via the portable control device 110. The instructions to the body position training device preferably include at least one of:
1. Feedback generation instructions based on a predetermined operative orientation of the body-mounted body position training device 300, such as a 45 degree bend of the neck, as shown in FIG. 6C.
2. Feedback generation instructions based on exceedance of a predetermined operative orientation threshold of the body-mounted body position training device 300, such as a bend of the neck exceeding 30 degrees as seen in FIG. 6B.

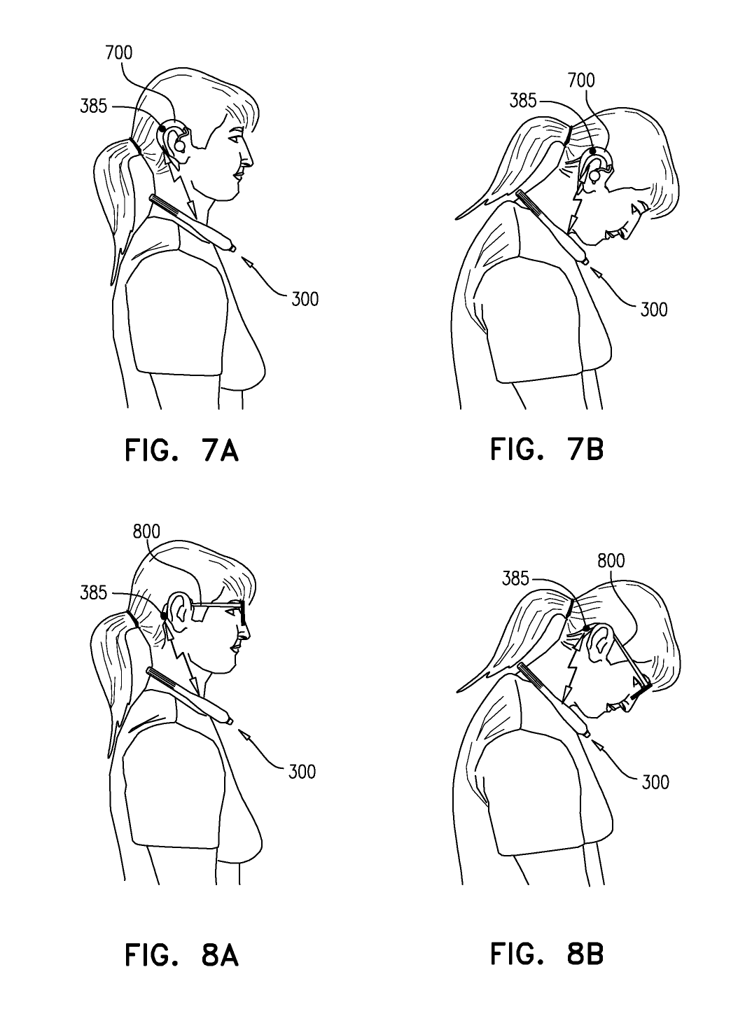

Reference is now made to FIGS. 7A and 7B, which are simplified illustrations of the body-mounted body position training device 300 as seen in FIG. 3. Orientation sensor 385 forms part of a hearing aid device 700 and is wirelessly connected to microprocessor 312 via wireless transceiver 318.

Reference is now made to FIGS. 8A and 8B, which are simplified illustrations of the body-mounted body position training device 300 as seen in FIG. 3. Orientation sensor 385 forms part of eyeglasses 800 and is wirelessly connected to microprocessor 312 via wireless transceiver 318.

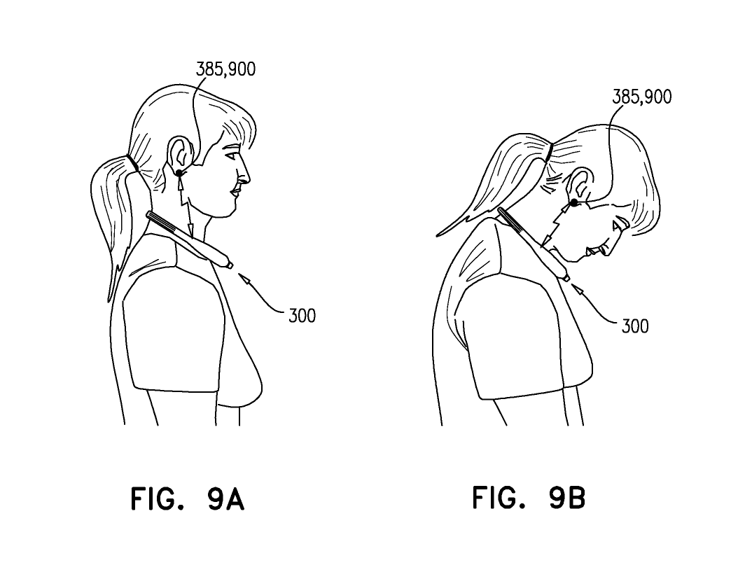

Reference is now made to FIGS. 9A and 9B, which are simplified illustrations of the body-mounted body position training device 300 as seen in FIG. 3. Orientation sensor 385 is attached to an earring 900 and is wirelessly connected to microprocessor 312 via a wireless transceiver 318.

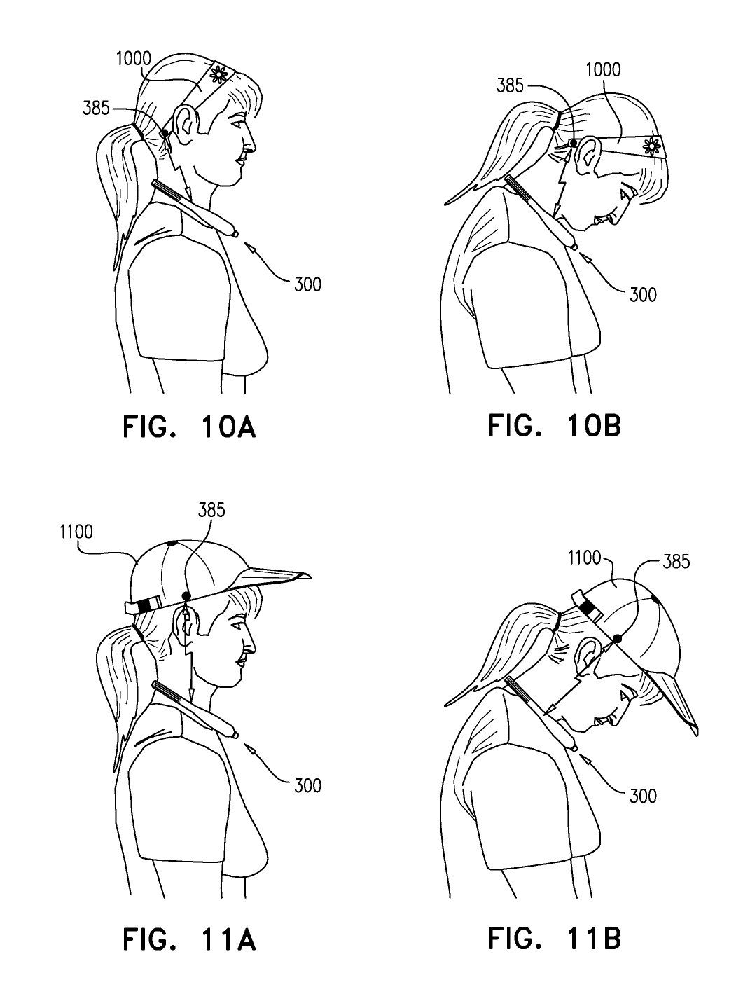

Reference is now made to FIGS. 10A and 10B, which are simplified illustrations of the body-mounted body position training device 300 as seen in FIG. 3. Orientation sensor 385 is attached to a hair-bow 1000 and is wirelessly connected to microprocessor 312 via wireless transceiver 318.

Reference is now made to FIGS. 11A and 11B, which are simplified illustrations of the body-mounted body position training device 300 as seen in FIG. 3. Orientation sensor 385 is attached to a hat 1100 and is wirelessly connected to microprocessor 312 via a wireless transceiver 318.

It will be appreciated by persons skilled in the art that the present invention is not limited by what has been particularly claimed and includes both combinations and subcombinations of features described and shown hereinabove as well as modifications thereof which are not in the prior art.

1. A body movement feedback system comprising:
at least one position sensor operative to sense in real time at least relative positions of mutually articulated body portions of a user and to provide sensor outputs;
a mutually articulated body portion position processor, receiving said sensor outputs of said at least one position sensor and providing processor outputs representing relative movements of the mutually articulated body portions; and
a real time user feedback generator operative to generate predetermined feedback to the user in response to predetermined selected movements of the mutually articulated body portions.

2. The body movement feedback system according to claim 1 and wherein said at least one position sensor is operative to sense at least relative orientations of at least one of the upper back, chest and neck relative to at least one of gravity and an orientation of a user's head.

3. The body movement feedback system according to claim 1 and also including a user feedback selector receiving feedback selection inputs from a person and operative to assign selectable user feedbacks to selectable movements of the mutually articulated body portions.

4. The body movement feedback system according to claim 1 and wherein said at least one position sensor includes at least two body portion orientation sensors.

5. The body movement feedback system according to claim 4 and wherein said at least two body portion orientation sensors include body portion orientation sensors attached to at least two of an upper back, a chest, a neck and a head of a user.

6. The body movement feedback system according to claim 1 and wherein said at least one position sensor includes at least one body portion orientation sensor attached to at least one of an upper back, a chest, a neck and a head of a user.

7. The body movement feedback system according to claim 1 and wherein said real time user feedback generator is operative to generate at least one of tactile feedback, auditory feedback and visual feedback.

8. The body movement feedback system according to claim 1 and wherein said at least one position sensor comprises at least one first sensor mounted at a first location onto at least one of a user's upper back, chest and neck by one of a necklace, a headset and a headphone.

9. The body movement feedback system according to claim 8 and wherein said at least one position sensor also comprises at least a second sensor mounted onto the user's head at a second location, said first and second locations being mutually separated.

10. The body movement feedback system according to claim 1 and wherein said real time user feedback generator comprises a feedback transducer operative to provide a real time notification to a user when at least one predetermined orientation is reached.

11. The body movement feedback system according to claim 1 and also comprising a portable controller communicating wirelessly with said processor.

12. A method for providing body movement feedback, the method comprising:
sensing in real time at least relative positions of mutually articulated body portions of a user and providing sensor outputs;
receiving said sensor outputs and providing processor outputs representing relative movements of the mutually articulated body portions; and
generating predetermined feedback to the user in response to predetermined selected movements of the mutually articulated body portions.

13. The method for providing body movement feedback according to claim 12 and wherein said relative positions comprise relative orientations of at least one of the upper back, chest and neck relative to at least one of gravity and an orientation of a user's head.

14. The method for providing body movement feedback according to claim 12 and also comprising:
receiving feedback selection inputs; and
selecting said predetermined feedback based on said selection inputs.

15. The method for providing body movement feedback according to claim 12 and wherein said sensing comprises sensing orientation of at least two of an upper back, a chest, a neck and a head of a user.

16. The method for providing body movement feedback according to claim 12 and wherein said predetermined feedback comprises at least one of tactile feedback, auditory feedback and visual feedback.

17. The method for providing body movement feedback according to claim 12 and wherein said predetermined feedback comprises a real time notification to a user when at least one predetermined orientation is reached.
